(19) United States
(12) Patent Application Publication
(10) Pub. No.: US 2022/0235329 A1
(43) Pub. Date: Jul. 28, 2022

(54) METHODS FOR GENERATING INDUCED PLURIPOTENT STEM CELLS VIA CELL CYCLE SYNCHRONIZATION

(71) Applicant: Orig3n, Inc., Boston, MA (US)

(72) Inventors: Robin Y. Smith, Boston, MA (US); Marcie A. Glicksman, Salem, MA (US); Nikhat F. Zaidi, Newton, MA (US)

(21) Appl. No.: 17/464,315

(22) Filed: Sep. 1, 2021

Related U.S. Application Data
(63) Continuation of application No. 15/965,313, filed on Apr. 27, 2018, now abandoned.
Application No. 15/965,313 is a continuation-in-part of application No. PCT/US16/59112, filed on Oct. 27, 2016.
(60) Provisional application No. 62/491,922, filed on Apr. 28, 2017.
(60) Provisional application No. 62/249,520, filed on Nov. 2, 2015.

(51) Int. Cl. C12N 5/074; G01N 33/52; G01N 15/14; B01L 3/00; G01N 15/04; C12M 1/00; C12N 5/00

(52) U.S. Cl. CPC C12N 5/0696; G01N 33/52; G01N 15/14; B01L 3/502761; G01N 15/1459; G01N 15/042; G01N 2015/1006; C12N 5/0018; B01L 2200/0652; C12N 2501/065; C12N 2501/36; C12N 2506/45; C12N 2501/15; C12M 47/04

(57) ABSTRACT

The techniques described herein provide for improved efficiency of iPSC production from biological cells. The approach achieves improved iPSC production efficiency by obtaining a set of cells whose cell cycles are synchronized at a specific, desired cell cycle phase, such as mitotic phase (also referred to as M phase). The efficacy with which such synchronized cells can be transformed into iPSCs is higher than for an arbitrary set of cells that comprises cells at a variety of different stages in their cycles. Accordingly, the approaches described herein allow efficient generation of iPSCs, thereby facilitating myriad technologies for personalized and regenerative medicine that rely upon the effective production of iPSCs.

### CROSS REFERENCE TO RELATED APPLICATIONS

This application is a continuation to U.S. Ser. No. 15/965,313, filed Apr. 27, 2018, which claims priority to and benefit of U.S. Provisional Patent Application No. 62/491,922, filed Apr. 28, 2017, and is a continuation-in-part of PCT Application No. PCT/US16/59112, filed Oct. 27, 2016, which claims priority to and benefit of U.S. Provisional Patent Application No. 62/249,520, filed Nov. 2, 2015. The entire contents of each of the foregoing applications are incorporated herein by reference in their entirety.

### BACKGROUND

Stem cells are rare, and difficult to isolate from adult tissues in appreciable numbers. In 2006 a research team led by Shinye Yamanaka published a paper describing how differentiated adult cells could be reprogrammed to display many of the properties of stem cells, including pluripotency (Takahashi, K. & Yamanaka, S. CELL 126:663-676 (2006)). These “induced pluripotent stem cells” (iPSCs) can replicate essentially indefinitely, and they can be differentiated into any of the cell types derived from the three embryonic germ layers to form potentially any cell in the human body. Thus, the development of iPSC methodologies has opened new avenues for personalized and regenerative medicine. Efficiencies of current methodologies for generating iPSCs are, however, low. Methods for increasing the efficiency of iPSC production would therefore be desirable.

### SUMMARY

The techniques described herein provide for improved efficiency of iPSC production from biological cells. The approach achieves improved iPSC production efficiency by obtaining a set of cells whose cell cycles are synchronized at a specific, desired cell cycle phase, such as mitotic phase (also referred to as M phase). The efficacy with which such synchronized cells can be transformed into iPSCs is higher than for an arbitrary set of cells that comprises cells at a variety of different stages in their cycles.

Synchronized cells are obtained using one or more of a variety of techniques described herein to arrest cell cycles of biological cells, followed by selection of a subset of the biological cells that are enriched in cells at the particular desired cell cycle phase. Accordingly, the approaches described herein allow efficient generation of iPSCs, thereby facilitating myriad technologies for personalized and regenerative medicine that rely upon the effective production of iPSCs.

In one aspect, the invention is directed to a method of generating a plurality of induced pluripotent stem cells (iPSCs) from a sample comprising a plurality of biological cells, the method comprising: (a) imposing one or more conditions on the biological cells of the sample for the purpose of arresting a cell cycle of the biological cells (e.g., a serum starvation condition; e.g., contact with a cell cycle inhibiting agent; e.g. incubation at a low temperature), wherein imposing the one or more conditions for arresting cell cycle comprises at least one of (i), (ii), and (iii): (i) incubating the biological cells in a medium comprising at least one of (A), (B), and (C): (A) a serum concentration that restricts cell growth [e.g., a serum concentration below a predetermined threshold concentration (e.g., less than 10%; e.g., less than about 5%; e.g., less than about 4%; e.g., less than about 3%; e.g., less than about 2%; e.g., less than about 1% serum concentration;); e.g., a serum concentration within a predetermined range (e.g., about 0% to about 10%; e.g., about 0.1% to about 5%; e.g., about 0.1% to about 2%; e.g., about 0.1% to about 1.0%); e.g., a serum free medium]; (B) a modified amino acid concentration that restricts cell grown [e.g., an amino acid concentration below a predetermined threshold concentration (e.g., less than 4 mM L-glutamine concentration); e.g., an amino acid concentration within a predetermined range]; and (C) a concentration of one or more particular carbohydrates (e.g., glucose) that restricts cell growth [e.g., a concentration of one or more particular carbohydrates (e.g., glucose) below a predetermined threshold concentration (e.g., less than 5.5 mM glucose concentration); e.g., a concentration of one or more particular carbohydrates within a predetermined range] such that the cells are arrested in their cell cycle (e.g., by incubating the biological cells in the medium for a predetermined amount of time and/or until the cell cycles of the biological cells has been determined to have been arrested); (ii) contacting the biological cells with a cell cycle inhibiting agent [e.g., a small molecule; e.g., a biomolecule; e.g., a reversible cell cycle inhibitor (e.g. the cell can restart its cell cycle after the agent is removed from media comprising the agent); e.g., a non-toxic agent (e.g., a concentration of the agent that is non-toxic to the cell is sufficient to arrest the cell cycle of the cell)] (e.g., by incubating the biological cells in a medium comprising the cell cycle inhibiting agent for a predetermined amount of time and/or until the cell cycles of the biological cells has been determined to have been arrested); and (iii) incubating the biological cells at a low temperature [e.g., a temperature below a predefined threshold temperature (e.g., less than about 35° C., less than 34° C., less than 33° C., less than 32° C., less than 31° C., or less than 30° C.); e.g., a temperature within a given predefined temperature range; e.g., a temperature of about 27° C., about 28° C., about 29° C., about 30° C., about 31° C., about 32° C., or about 33° C.] for a predetermined amount of time and/or until the cell cycles of the biological cells has been determined to have been arrested; (b) following step (a), selecting, from the biological cells, a subset of cells (e.g., separating the selected subset of cells from the remaining biological cells) determined [e.g., based on a property of the cells such as quantity of DNA within the cells, a size of the cells, a shape of the cells; e.g., via a cell sorting method (e.g., using a marker indicative of a specific cell cycle phase; e.g., using FACS, e.g., using centrifugal elutriation; e.g., using mitotic shake-off)] to be at a specific desired cell cycle phase, thereby obtaining a subset of cells that are enriched in cells at the same specific desired cell cycle phase (e.g., interphase; e.g., G0 phase; e.g., G0/G1 phase; e.g., early G1 phase; e.g., G1 phase; e.g., late G1 phase; e.g., G1/S phase; e.g., S phase; e.g., G2/M phase; e.g., M phase); and (c) delivering to the selected subset of cells (e.g., having been separated from the remaining biological cells) one or more transformation agents (e.g., one or more transformation agents that, when delivered to a cell, transforms the cell into an iPSC), thereby obtaining a plurality of iPSCs.

In certain embodiments, the method comprises imposing the one or more conditions to arrest the cell cycle at a first cell cycle phase (e.g., G1/S phase, as in following contact with thymidine for 16-24 hours), and then removing the one or more conditions for a predefined amount of time (e.g., 12 hours) or until the specific desired cell cycle phase is determined to have been reached [e.g., (I) removing the one or more conditions on the cells (e.g., ceasing contact of the cells with thymidine) prior to selection of the subset of cells in step (b), or (II) continuing to impose the one or more conditions on the cells during the selection of the subset of cells in step (b) and removing the one or more conditions on the subset of cells following selection of the subset in step (b)], such that the selected subset of cells is in a second cell cycle phase (e.g., M phase; e.g., a second cell cycle phase different from the first cell cycle phase) when step (c) is performed.

In certain embodiments, imposing the one or more conditions to arrest the cell cycle at the first cell cycle phase comprises incubating the biological cells in a first medium for a predetermined amount of time or until the first cell cycle phase has been determined to have been reached, and removing the one or more conditions comprises at least one of (I) and (II): (I) incubating the biological cells in a second medium for a predefined amount of time or until the second cell cycle phase is determined to have been reached, prior to selection of the subset of cells in step (b); and (II) incubating the selected subset of cells in a second medium for a predefined amount of time or until the second cell cycle phase is determined to have been reached, following selection of the subset of cells in step (b).

In certain embodiments, the first medium is a medium comprising at least one of (A), (B), and (C): (A) a serum concentration that restricts cell growth, (B) a modified amino acid concentration that restricts cell grown, and (C) a concentration of one or more particular carbohydrates (e.g., glucose) that restricts cell growth, and the second medium is a medium comprising at least one of (A), (B), and (C): (A) a serum concentration that allows cell growth [e.g., a serum concentration that is higher than the serum concentration of the first medium; e.g., a serum concentration below a predetermined threshold concentration (e.g., greater than or equal to 10%); e.g., a serum concentration within a predetermined range (e.g., about 2% to about 35%; e.g., about 5% to about 30%; e.g., about 10% to about 25%), (B) an amino acid concentration that allows cell grown [e.g., an amino acid concentration that is higher than an amino acid concentration of the first medium; e.g., an amino acid concentration above a predetermined threshold concentration (e.g., greater than or equal to 4 mM L-glutamine concentration); e.g., an amino acid concentration within a predetermined range], and (C) a concentration of one or more particular carbohydrates (e.g., glucose) that allows cell growth [e.g., a glucose concentration that is higher than a glucose concentration of the first medium; e.g., a concentration of one or more particular carbohydrates (e.g., glucose) above a predetermined threshold concentration (e.g., greater than or equal to 5.5 mM glucose concentration); e.g., a concentration of one or more particular carbohydrates within a predetermined range].

In certain embodiments, the first medium comprises a cell cycle inhibiting agent at a concentration sufficient to arrest the cell cycle of the biological cells, and the second medium does not comprise the cell cycle inhibiting agent at a concentration sufficient to arrest the cell cycle of cells (e.g., the biological cells; e.g., the selected subset of cells).

In certain embodiments, step (a) comprises contacting the biological cells with an agent for the purpose of modulating at least one pathway selected from the group consisting of a CEK interacting protein (cip) pathway, kinase inhibitory protein (kip) pathway, inhibitor of kinase 4 (INK4a) pathway, and an alternative reading frame (ARF) pathway. In certain embodiments, the agent modulates at least one protein selected from the group consisting of p14^{ARF}, p16^{INK4a}, p18, p19, p21, p27, p53, and p57. In certain embodiments, the agent comprises Transforming Growth Factor β (TGFβ).

In certain embodiments, step (a) comprises contacting the biological cells with an agent for the purpose of modulating a cyclin D pathway [e.g., a cyclin dependent kinase inhibitor (e.g., selected from the group consisting of palbociclib, ribociclib, voruciclib, and abemaciclib); e.g., a cyclin dependent kinase 4 (CDK4) inhibitor; e.g., a cyclin dependent kinase 6 (CDK6) inhibitor].

In certain embodiments, step (a) comprises contacting the biological cells with an agent for the purpose of inhibiting nucleotide biosynthesis (e.g., including synthetic or natural analogs of intermediates in the synthetic pathway or identified inhibitors of the catalytic activity, e.g., hydroxyurea; e.g., thymidine; e.g., thereby arresting cells at the transition between G1 phase and S phase).

In certain embodiments, step (a) comprises contacting the biological cells with an agent for the purpose of inhibiting microtubule polymerization (e.g., nocodazole).

In certain embodiments, step (a) comprises contacting the biological cells with an agent for the purpose of inhibiting HMG CoA reductase (e.g., a HMG CoA reductase inhibitor; e.g., lovastatin).

In certain embodiments, step (a) comprises contacting the biological cells with a DNA polymerase inhibitor (e.g., aphidicolin).

In certain embodiments, step (a) comprises contacting the biological cells with an agent selected from the group consisting of abemaciclib, aminopterin, aphidicolin, blebbistatin, butyrate, cathinone, colcemid, colchicine, compactin, cytochalasin D, cytosine arabinoside, fluorodeoxyuridine, hydroxyurea, lovastatin, methotrexate, mevinolin, MG132, mimosine, nocodazole, noscapine, palbociclib, pantopon, razoxane, reveromycin A, RO-3306, roscovitine, ribociclib, vincristine, or voruciclib.

In certain embodiments, the specific desired cell cycle phase is M phase.

In certain embodiments, step (b) comprises: contacting the plurality of biological cells with a labeled agent, the labeled agent comprising a detectable dye [e.g., a dye that absorbs light at one or more particular wavelengths (e.g., infrared wavelengths; e.g., visible wavelengths; e.g., ultraviolet wavelengths); e.g., a fluorescent dye that emits light at one or more particular wavelengths]; detecting a signal indicative of the presence and/or quantity of the labeled agent within and/or on the biological cells based on the detectable dye (e.g., detecting absorption of light by the biological cells at one or more particular wavelengths at which the dye absorbs light; e.g., detecting a fluorescent signal emitted from the biological cells); and selecting, as the subset of cells, biological cells that are determined to comprise a substantially similar quantity of the labeled agent based on the detected signal (e.g., selecting cells for which a similar absorption signal is detected; e.g., selecting cells for which a similar fluorescent signal is detected).

In certain embodiments, the labeled agent binds to nucleic acids (e.g., double stranded nucleic acids; e.g., DNA). In certain embodiments, the labeled agent binds to chromatin. In certain embodiments, the labeled agent binds to microtubules. In certain embodiments, the labeled agent binds to a cell surface marker (e.g., a particular protein on a surface of the cell). In certain embodiments, the labeled agent binds to or comprises a cellular marker for proliferation.

In certain embodiments, the labeled agent binds to Ki-67 (e.g., wherein the labeled agent comprises an antibody that binds to Ki-67) [e.g., wherein Ki67 is preferentially expressed during the M phase, late G1 phase, S phase, and/or G2 phase, while cells in the G0 phase (non-cycling) have low or no Ki67 expression, wherein the labeled agent comprises an antibody that binds to (e.g., recognizes) Ki-67, thereby identifying cells that are in the M phase, late G1 phase, S phase, and/or G2 phase, as opposed to the G0 (non-cycling) phase].

In certain embodiments, the labeled agent binds to histone H3 pSer28 (e.g., wherein the labeled agent comprises an antibody that binds to histone H3 pSer28).

In certain embodiments, the labeled agent binds to Bromodeoxyuridine (BrdU) (e.g., wherein the labeled agent comprises an antibody that binds to BrdU; e.g., wherein BrdU is introduced into the biological cells such that replicating cells incorporate BrdU into newly synthesized DNA during replication, such that binding of the labeled agent to BrdU is indicative of cells that are replicating (e.g., cells that are in S phase)).

In certain embodiments, the labeled agent comprises a dye that is initially nonfluorescent and becomes florescent following cleavage by esterase within a cell, thereby allowing detection of cellular proliferation (e.g., wherein the labeled agent comprises Violet Proliferation Dye 450).

In certain embodiments, the labeled agent comprises an antibody that binds to cyclin E thereby identifying cells that are in the S-phase. In certain embodiments, the labeled agent comprises an antibody that binds to cyclin B1 thereby identifying cells that are in (or moving towards) the G2/M-phase.

In certain embodiments, the method comprises using fluorescence activated cell sorting to select the subset of cells.

In certain embodiments, step (b) comprises: detecting a scattering signal indicative of an amount of light scattered by each of at least a portion of the biological cells (e.g., a forward scattering signal; e.g., a side scattering signal); and selecting, as the subset of cells, biological cells determined to be in a same cell cycle phase based on the detected scattering signal [e.g., selecting biological cells for which the detected scattering signal is substantially similar (e.g., within a predefined range; e.g., above or below a predefined threshold signal)].

In certain embodiments, step (b) comprises using centrifugation elutriation to select the subset of cells. In certain embodiments, step (b) comprises using mitotic shake-off to select the subset of cells.

In certain embodiments, at least one of the one or more transformation agents comprises a gene selected from the group consisting of an Oct family gene, a Klf family gene, a Sox family gene, a Myc family gene, a Lin family gene, and a Nanog gene.

In certain embodiments, at least one of the one or more transformation agents comprises a gene selected from the group consisting of Oct3/4, Oct4, Klf4, Klf1, Klf2, Klf5, Sox2, Sox1, Sox3, Sox15, Sox17, Sox18, c-Myc, L-Myc, N-Myc, TERT, SV40 Large T antigen, HPV16 E6, HPV16 E7, Bmil, Lin28, Lin28b, Nanog, Glis1, Esrrb, and Esrrg.

In certain embodiments, at least one of the one or more transformation agents comprises a gene selected from the group consisting of Klf4, Oct-3/4, Oct-4, Sox2, and c-Myc.

In certain embodiments, step (c) comprises transducing the subset of cells with the gene. In certain embodiments, the method comprises using a vector comprising the gene to transduce the subset of cells with the gene. In certain embodiments, the vector comprises at least one of a plasmid, a virus, a transposable element, and a nanoparticle. In certain embodiments, the vector comprises a virus, and the virus is a Sendai virus or an adenovirus.

In certain embodiments, at least one of the one or more transforming agents is selected from the group consisting of valproic acid, BIX-01294, SB431412, or PD0325901.

In certain embodiments, at least one of the one or more transforming agents is selected from the group consisting of a glycogen synthase kinase inhibitor, TGFP receptor inhibitor, cyclic AMP agonist, S-adenosyl homocysteine hydrolase inhibitor, and agent that promotes histone acetylation.

In certain embodiments, at least one of the one or more transformation agents is a microRNA.

In certain embodiments, the biological cells are somatic cells. In certain embodiments, the biological cells are mammalian cells. In certain embodiments, the biological cells are human cells.

In certain embodiments, the method further comprises differentiating the plurality of iPSCs into one or more types of cells. In certain embodiments, at least one of the one or more types of cells is selected from the group consisting of fibroblasts, B cells, T cells, hematopoietic cells, macrophages, monocytes, mononuclear cells, dendritic cells, myocytes, keratinocytes, melanocytes, adipocytes, epithelial cells, epidermal cells, chondrocytes, neural cells, glial cells, astrocytes, cardiac cells, cardiomyocytes, esophageal cells, gastric cells, pancreatic cells, hepatocytes, cumulus cells, and gametocytes.

In some aspects, the invention relates to a method for generating an induced pluripotent stem cell (iPSC), comprising: providing a cell; arresting the cell cycle of the cell; and transforming the cell, thereby generating the induced pluripotent stem cell. This method may also be used to generate a plurality of induced pluripotent stem cells.

In certain embodiments, arresting the cell cycle comprises incubating the cell in media comprising a serum concentration or amino acid concentration that restricts cell growth.

In certain embodiments, arresting the cell cycle comprises modulating a CEK interacting protein (cip) pathway, kinase inhibitory protein (kip) pathway, inhibitor of kinase 4 (INK4a) pathway, or alternative reading frame (ARF) pathway.

In certain embodiments, arresting the cell cycle comprises activating p14ARF, p16INK4, p21, p27, p53, or p57.

In certain embodiments, wherein arresting the cell cycle comprises modulating a cyclin D pathway.

In certain embodiments, arresting the cell cycle comprises inhibiting cyclin dependent kinase 4 or cyclin dependent kinase 6.

In certain embodiments, arresting the cell cycle comprises contacting the cell with a cyclin dependent kinase inhibitor. In certain embodiments, the cyclin dependent kinase inhibitor is a cyclin dependent kinase 4 inhibitor or a cyclin dependent kinase 6 inhibitor. In certain embodiments, the cyclin dependent kinase inhibitor is palbociclib, ribociclib, voruciclib, or abemaciclib.

In certain embodiments, arresting the cell cycle comprises incubating the cell in media comprising thymidine.

In certain embodiments, arresting the cell cycle comprises inhibiting microtubule polymerization in the cell. In certain embodiments, inhibiting microtubule polymerization comprises contacting the cell with an inhibitor of microtubule polymerization.

In certain embodiments, arresting the cell cycle comprises inhibiting nucleotide biosynthesis in the cell. In certain embodiments, inhibiting nucleotide biosynthesis in the cell comprises contacting the cell with an inhibitor of nucleotide biosynthesis.

In certain embodiments, arresting the cell cycle comprises inhibiting HMG CoA reductase in the cell. In certain embodiments, inhibiting HMG CoA reductase comprises contacting the cell with an HMG CoA reductase inhibitor.

In certain embodiments, arresting the cell cycle comprises inhibiting DNA polymerase in the cell. In certain embodiments, inhibiting DNA polymerase comprises contacting the cell with a DNA polymerase inhibitor.

In certain embodiments, arresting the cell cycle comprises contacting the cell with lovastatin, compactin, mevinolin, mimosine, aphidicolin, aminopterin, hydroxyurea, colchicine, colcemid, razoxane, roscovitine, vincristine, cathinone, pantopon, aminopterin, methotrexate, fluorodeoxyuridine, butyrate, cytosine arabinoside, MG132, RO-3306, noscapine, blebbistatin, reveromycin A, cytochalasin D, ornocodazole.

In certain embodiments, arresting the cell cycle comprises incubating the cell at low temperature. In certain embodiments, the cell is incubated cell at about 27° C. to about 33° C.

In certain embodiments, arresting the cell cycle comprises arresting the cell cycle at interphase, G0 phase, G0/G1 phase, early G1 phase, G1 phase, late G1 phase, G1/S phase, S phase, G2/M phase, or M phase. In certain embodiments, the cell cycle is arrested at M phase.

In certain embodiments, a plurality of induced pluripotent stem cells are generated.

In certain embodiments, transforming the cell(s) comprises transducing the cell(s) with at least one gene selected from the group consisting of an Oct family gene, a Klf family gene, a Sox family gene, a Myc family gene, a Lin family gene, and a Nanog gene.

In certain embodiments, transforming the cell(s) comprises transducing the cell(s) with at least one gene selected from the group consisting of Oct3/4, Oct4, Klf4, Klf1, Klf2, Klf5, Sox2, Sox1, Sox3, Sox15, Sox17, Sox18, c-Myc, L-Myc, NMyc, TERT, SV40 Large T antigen, HPV16 E6, HPV16 E7, Bmil, Lin28, Lin28b, Nanog, Glis1, Esrrb, and Esrrg.

In certain embodiments, transforming the cell(s) comprises transducing the cell(s) with at least one gene selected from the group consisting of Klf4, Oct-3/4, Oct-4, Sox2, and c-Myc.

In certain embodiments, transducing the cell(s) comprises transducing the cell(s) with a vector comprising at least one gene. In certain embodiments, the vector comprises a plasmid, virus, transposable element, or nanoparticle. In certain embodiments, the vector comprises a virus, and the virus is a Sendai virus or an adenovirus.

In certain embodiments, transforming the cell(s) comprises contacting the cell(s) with valproic acid, BIX-01294, SB431412, or PD0325901.

In certain embodiments, transforming the cell(s) comprises contacting the cell(s) with at least one of a glycogen synthase kinase inhibitor, TGFP receptor inhibitor, cyclic AMP agonist, S-adenosyl homocysteine hydrolase inhibitor, and agent that promotes histone acetylation.

In certain embodiments, the cell(s) are somatic cell(s). In certain embodiments, the cell(s) are mammalian cell(s). In certain embodiments, the cell(s) are human cell(s).

In some aspects, the invention relates to a method for generating a plurality of induced pluripotent stem cells, comprising: providing a plurality of cells; selecting a subset of the plurality of cells, wherein the subset of cells is enriched in cells of one or more cell cycle phases; and transforming the subset of cells, thereby generating the plurality of induced pluripotent stem cells.

In certain embodiments, the subset of cells are enriched in G0 phase, G0/G1 phase, early G1 phase, G1 phase, late G1 phase, G1/S phase, S phase, G2/M phase, or M phase.

In certain embodiments, the method comprises contacting the plurality of cells with a dye that binds to nucleic acids, wherein selecting the subset of cells comprises selecting cells that comprise a similar amount of the dye.

In certain embodiments, the cells are selected by fluorescence activated cell sorting. In certain embodiments, the cells are selected by centrifugation elutriation or mitotic shake-off.

In certain embodiments, transforming the cell(s) comprises transducing the cell(s) with at least one gene selected from the group consisting of an Oct family gene, a Klf family gene, a Sox family gene, a Myc family gene, a Lin family gene, and a Nanog gene.

In certain embodiments, transforming the cell(s) comprises transducing the cell(s) with at least one gene selected from the group consisting of Oct3/4, Oct4, Klf4, Klf1, Klf2, Klf5, Sox2, Sox1, Sox3, Sox15, Sox17, Sox18, c-Myc, L-Myc, NMyc, TERT, SV40 Large T antigen, HPV16 E6, HPV16 E7, Bmil, Lin28, Lin28b, Nanog, Glis1, Esrrb, and Esrrg.

In certain embodiments, transforming the cell(s) comprises transducing the cell(s) with at least one gene selected from the group consisting of Klf4, Oct-3/4, Oct-4, Sox2, and c-Myc.

In certain embodiments, transducing the cell(s) comprises transducing the cell(s) with a vector comprising at least one gene. In certain embodiments, the vector comprises a plasmid, virus, transposable element, or nanoparticle. In certain embodiments, the vector comprises a virus, and the virus is a Sendai virus or an adenovirus.

In certain embodiments, transforming the cell(s) comprises contacting the cell(s) with valproic acid, BIX-01294, SB431412, or PD0325901.

In certain embodiments, transforming the cell(s) comprises contacting the cell(s) with at least one of a glycogen synthase kinase inhibitor, TGFP receptor inhibitor, cyclic AMP agonist, S-adenosyl homocysteine hydrolase inhibitor, and agent that promotes histone acetylation.

In certain embodiments, the cell(s) are somatic cell(s). In certain embodiments, the cell(s) are mammalian cell(s). In certain embodiments, the cell(s) are human cell(s).

Details described with respect to one aspect of the invention may be applied to other aspects of the invention.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a block diagram showing a process for generating a plurality of induced pluripotent stem cells (iPSCs) from a sample comprising a plurality of biological cells, according to an illustrative embodiment.

FIG. 2 is a block diagram showing a process for generating an induced pluripotent stem cell (iPSC) from a cell, according to an illustrative embodiment.

FIG. 3 is a block diagram showing a process for generating a plurality of induced pluripotent stem cells (iPSCs) from a sample comprising a plurality of biological cells, according to an illustrative embodiment.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted.

Headers are provided for the convenience of the reader—the presence and/or placement of a header is not intended to limit the scope of the subject matter described herein.

In certain embodiments, the methods described herein are directed to an approach wherein synchronizing the cell cycle of a group of cells results in a higher yield of induced pluripotent stem cells (iPSCs) following subsequent transformation steps. Synchronization may be accomplished, for example, by arresting the cell cycle of a cell, or by selecting cells enriched in one or more cell cycle phases from a plurality of cells.

In some aspects, the invention relates to a method for generating an induced pluripotent stem cell, comprising: providing a cell; arresting the cell cycle of the cell; and transforming the cell, thereby generating the induced pluripotent stem cell. The cell is preferably not a stem cell (e.g., the cell may be a differentiated cell). This method may also be used to generate a plurality of induced pluripotent stem cells.

In some aspects, the invention relates to a method for generating a plurality of induced pluripotent stem cells, comprising: providing a plurality of cells; selecting a subset of the plurality of cells, wherein the subset of cells is enriched in cells of one or more cell cycle phases; and transforming the subset of cells, thereby generating the plurality of induced pluripotent stem cells. In preferred embodiments, the cells are not stem cells. The plurality of cells may comprise stem cells, and such a plurality would also comprise cells that are not stem cells (e.g., the plurality would also comprise differentiated cells).

### I. Cells

The cell may be a eukaryotic cell, such as a metazoan cell. The cell may be a mammalian cell. The cell may be from a rodent, lagomorph, feline, canine, porcine, ovine, bovine, equine, or primate. For example, the cell may be a human cell. In preferred embodiments, the cell is a somatic cell. In certain embodiments, the cell is a diploid cell.

The cell may be a differentiated cell. The cell may be derived from the ectoderm, endoderm, or mesoderm. The cell may originate from the epithelium, connective tissue, muscle tissue, or nervous tissue. The cell may be a peripheral blood mononuclear cell (PBMC) or a fibroblast. The cell may be a lymphocyte. In some embodiments, the cell is an adipocyte.

### II. Arresting the Cell Cycle

In certain embodiments, the methods described herein comprise arresting the cell cycle of the cell. Arresting the cell cycle of the cell may comprise any method that inhibits mitosis (see, e.g., BANFALVI, GASPAR, CELL CYCLE SYNCHRONIZATION (Humana Press, 2011); and PCT Patent Application Publication No. WO 2010/118709 (hereby incorporated by reference)). Cells that are in the mitosis phase of the cell cycle (M phase) are more receptive to transduction and/or transfection. Thus, in some certain embodiments, arresting the cell cycle comprises arresting the cell cycle at M phase (e.g., by contacting the cell with an agent such as colchicine, colcemid, razoxane, or noscapine). Nevertheless, arresting the cell cycle may comprise arresting the cell cycle at interphase, such as G0 phase, G1 phase, S phase, or G2 phase. For example, a cell may be arrested in G1/S phase (e.g., using thymidine, such as 4 mM thymidine for 16-24 hours), and the method may comprise incubating the cell for a period of time after releasing the cell from arrest, prior to transforming the cell (e.g., 12 hours after release from a thymidine block, such that the cell is in M phase when it is transformed).

A method may comprise arresting the cell cycle of a plurality of cells, and for such embodiments, the phrase “arresting the cell cycle” is synonymous with “synchronizing the cell cycle” or “cell synchronization”.

In some embodiments, cell synchronization may be achieved by using replication inhibitors using compounds such as hydroxyurea, thymidine and amphidicolin. These compounds may inhibit replication through different pathways. For example, while hydroxyurea and thymidine cause a decrease in free deoxyribonucleotide triphosphate (dNTP), which is the main structural unit of DNA, amphidicolin is a direct inhibitor of DNA polymerases. As described previously, the inhibition of DNA replication, for example using thymidine, synchronizes the cells in between the G1 phase, where the cell is in preparation for replication, and the S phase, where the replication occurs.

In some embodiments, arresting the cell cycle does not halt the cell cycle at a specific phase, and yet the cell cycle is inhibited such that transformation is more efficient than without arresting the cell cycle. For example, aphidicolin and nocodazole may be used to arrest a cell at G2/M phase, which is useful to increase transformation efficiency. Arresting the cell cycle may comprise arresting the cell cycle at interphase, G0 phase, G0/G1 phase, early G1 phase, G1 phase, late G1 phase, G1/S phase, S phase, G2/M phase, or M phase.

Arresting the cell cycle or cell synchronization by serum starvation or nutrient starvation may be used to arrest the cell cycle, for example, in G1 phase or G0 phase (see, e.g., U.S. Pat. No. 8,993,328; hereby incorporated by reference). Serum starvation causes the cells to cease their growth, the effects of which are observed at the start of the DNA replication stage in the cells (G1 phase), thereby causing the cells to synchronize at this phase. In some embodiments, arresting the cell cycle comprises incubating the cell in media comprising a serum concentration and/or amino acid concentration that restricts cell growth. The cell may be incubated in media comprising a serum concentration and/or amino acid concentration that restricts cell growth for about 1 hour to about 10 days, such as about 1 day to about 7 days, such as about 1, 2, 3, 4, 5, 6, or 7 days. The period of time may depend on different factors, e.g., because different cells and different cell culture conditions result in cell cycles of varying duration. The media may have a serum concentration, for example, of about 0% to about 10%, such as about 0.1% to about 5%, about 0.1% to about 2%, or about 0.1% to about 1.0%. The media may have a serum concentration of less than about 5%, less than about 4%, less than about 3%, less than about 2%, or less than about 1%. The media may have a serum concentration of about 0.1%, about 0.2%, about 0.3%, about 0.4%, about 0.5%, about 0.6%, about 0.7%, about 0.8%, about 0.9%, or about 1.0%. The media may be serum free media (i.e., media comprising no serum). The cell cycle may then be restarted, for example, by contacting the cell with media comprising a second serum concentration (i.e., a higher serum concentration than the serum concentration that restricts cell growth). Thus, the method may comprise incubating the cell in media comprising a second serum concentration and/or a second amino acid concentration, e.g., wherein the second serum concentration and/or second amino acid concentration is higher than the serum concentration or amino acid concentration that restricts cell growth. The second serum concentration may be about 2% to about 35%, such as about 5% to about 30%, or about 10% to about 25%. The second serum concentration may be about 10%, about 11%, about 12%, about 13%, about 14%, about 15%, about 16%, about 17%, about 18%, about 19%, about 20%, about 21%, about 22%, about 23%, about 24%, or about 25%. The serum may be, for example, fetal bovine serum.

Arresting the cell cycle may comprise contacting the cell with an agent. Contacting the cell with an agent may comprise incubating the cell in media comprising the agent. The agent may be a small molecule or a biomolecule. As used here, the term “small molecule” refers to molecules with a molecular weight less than 5,000 amu, such as about 30 to about 1000 amu, about 40 amu to about 800 amu, or about 50 amu to about 750 amu. The term “biomolecule” refers to molecules comprising peptides, proteins, nucleic acids, sugars, and/or carbohydrates. A biomolecule may be, for example, a cytokine (e.g., Transforming Growth Factor β). The agent may be an inhibitor of a transcription factor, enzyme (e.g., a kinase or phosphorylase), or cellular pathway. For example, the agent may be a cyclin dependent kinase inhibitor, a cyclin dependent kinase 4 inhibitor, a cyclin dependent kinase 6 inhibitor, a DNA polymerase inhibitor, a HMG CoA reductase inhibitor, an inhibitor of nucleotide biosynthesis, or an inhibitor of microtubule polymerization.

In certain embodiments, the agent is a reversible inhibitor of the cell cycle, e.g., the cell can restart its cell cycle after the agent is removed from media comprising the agent. In certain embodiments, the agent is non-toxic, e.g., a concentration of the agent that is non-toxic to the cell is sufficient to arrest the cell cycle of the cell. In certain embodiments, incubating the cell in media comprising the agent comprises incubating the cell in media comprising a concentration of the agent that is not toxic to the cell. The agent may be abemaciclib, aminopterin, aphidicolin, blebbistatin, butyrate, cathinone, colcemid, colchicine, compactin, cytochalasin D, cytosine arabinoside, fluorodeoxyuridine, hydroxyurea, lovastatin, methotrexate, mevinolin, MG132, mimosine, nocodazole, noscapine, palbociclib, pantopon, razoxane, reveromycin A, RO-3306, roscovitine, ribociclib, vincristine, or voruciclib.

Incubating the cell in media comprising an agent may comprise incubating the cell in media comprising the agent for about 1 hour to about 10 days, such as about 2 hours to about 7 days, such as about 1, 2, 3, 4, 5, 6, or 7 days. The period of time may depend on different factors, e.g., because different cells and different cell culture conditions result in cell cycles of varying duration.

In some embodiments, the method comprises incubating the cell in media that does not comprise the agent, e.g., after incubating the cell in media comprising the agent, to restart the cell cycle. For example, the method may comprise incubating the cell in media that does not comprise the agent for about 4 hours to about 24 hours following a double thymidine block, e.g., such that the cell is in M phase during transformation.

In some embodiments, arresting the cell cycle comprises modulating a CEK interacting protein (cip) pathway, kinase inhibitory protein (kip) pathway, inhibitor of kinase 4 (INK4a) pathway, or alternative reading frame (ARF) pathway. For example, the cip/kip family members p21, p27, and p57 arrest the cell cycle at G1 phase. Transforming Growth Factor β (TGFβ) may be used to activate p27. Similarly, the INK4a/ARF family includes p16^{INK4a}, which binds to cyclin dependent kinase 4 (CDK4) to arrest the cell cycle at G1 phase. In some embodiments, arresting the cell cycle comprises activating p14^{ARF}, p16^{INK4a}, p18, p19, p21, p27, p53, or p57 (see, e.g., U.S. Pat. No. 6,033,847; hereby incorporated by reference).

In some embodiments, arresting the cell cycle comprises modulating a cyclin D pathway. Arresting the cell cycle may comprise inhibiting cyclin dependent kinase 4 (CDK4) or cyclin dependent kinase 6 (CDK6). Arresting the cell cycle may comprise contacting the cell with a cyclin dependent kinase inhibitor. The cyclin dependent kinase inhibitor may be, for example, a cyclin dependent kinase 4 inhibitor or a cyclin dependent kinase 6 inhibitor. Palbociclib is a selective inhibitor of both CDK4 and CDK6. In some embodiments, the cyclin dependent kinase inhibitor is palbociclib, ribociclib, voruciclib, or abemaciclib (see also PCT Patent Application Publication No. WO 2014/109858; hereby incorporated by reference).

In some embodiments, arresting the cell cycle comprises inhibiting nucleotide biosynthesis in the cell. Inhibiting nucleotide biosynthesis in the cell comprises contacting the cell with an inhibitor of nucleotide biosynthesis.

In some embodiments, arresting the cell cycle comprises incubating the cell in media comprising hydroxyurea or thymidine. Hydroxyurea and thymidine cause the decrease of free deoxyribonucleotide triphosphates (dNTPs), the main structural units of DNA. The inhibition of DNA replication arrests cells on the transition between the G1 phase and S phase. The advantages of this method include the potential to reach a homogenous synchronized cell population by repeated exposure to the replication inhibitors and the fact that the replication inhibitors are inexpensive.

In some embodiments, arresting the cell cycle comprises inhibiting microtubule polymerization in the cell. Inhibiting microtubule polymerization may comprise contacting the cell with an inhibitor of microtubule polymerization, such as nocodazole.

In some embodiments, arresting the cell cycle comprises inhibiting HMG CoA reductase in the cell. Inhibiting HMG CoA reductase may comprise contacting the cell with an HMG CoA reductase inhibitor, such as lovastatin.

In some embodiments, arresting the cell cycle comprises inhibiting DNA polymerase in the cell. Inhibiting DNA polymerase may comprises contacting the cell with a DNA polymerase inhibitor, such as aphidicolin.

In some embodiments, arresting the cell cycle comprises contacting the cell with abemaciclib, aminopterin, aphidicolin, blebbistatin, butyrate, cathinone, colcemid, colchicine, compactin, cytochalasin D, cytosine arabinoside, fluorodeoxyuridine, hydroxyurea, lovastatin, methotrexate, mevinolin, MG132, mimosine, nocodazole, noscapine, palbociclib, pantopon, razoxane, reveromycin A, RO-3306, roscovitine, ribociclib, vincristine, or voruciclib.

In some embodiments, arresting the cell cycle comprises incubating the cell at low temperature. The low temperature may be, for example, less than about 35° C., less than 34° C., less than 33° C., less than 32° C., less than 31° C., or less than 30° C. The low temperature may be about 20° C. to about 35° C., such as about 27° C. to about 33° C. The low temperature may be about 27° C., about 28° C., about 29° C., about 30° C., about 31° C., about 32° C., or about 33° C. The cell may be incubated in at low temperature for about 1 hour to about 10 days, such as about 1 day to about 7 days, such as about 1, 2, 3, 4, 5, 6, or 7 days. The period of time may depend on different factors, e.g., because different cells and different cell culture conditions result in cell cycles of varying duration.

In certain embodiments, arresting the cell cycle or cell synchronization is achieved by subjecting the mitotic cells to vibration. For example, cell synchronization in the late telophase may be achieved by subjecting mitotic cells in a culture bottle with culture medium to vibration under standard culture conditions (e.g., 37° C., 100% humidity, 5% CO₂, etc.) in an incubator. These cells may be attached or adherent cells. The vibration of the culture bottle containing the adherent cells may wash-out or dislodge the poorly adherent mitotic cells into the culture medium, which results in inhibition of cell growth of the unanchored cells. For example, the washed-out unanchored mitotic cells may proceed until karyokinesis (nuclear segmentation), and not complete cytokinesis or final cell division, or late telophase. In certain embodiments, the vibration may be in the frequency range of 1 to 100 Hz, and the amplitude of the vibration may be in the range of 0.001 mm to 30 mm. In certain embodiments, the mitotic cells are subjected to the vibration treatment for a period of time in the range of 1 to 30 hours, or 1 day. In certain embodiments, other treatments (e.g., a washout agent) may be added to the culture media of the cells to aide in the dislodging of cells during the vibration treatment.

In certain embodiments, the method comprises arresting the cell cycle using more than one of the methods described herein, including serum starvation or nutrient starvation, contacting the cell with an agent, contacting the cell with more than one agent, modulating a cellular pathway, incubating the cell at low temperature, and subjecting the cells to vibration.

### III. Selecting Cells at a Phase of the Cell Cycle

In some embodiments, the approach described herein comprises selecting a subset of the plurality of cells, wherein the subset of cells is enriched in cells of one or more cell cycle phases. The methods may further comprise arresting the cell cycle for the cells of the plurality, e.g., prior to selecting the subset, using any method described herein. Similarly, the methods may further comprise arresting the cell cycle for the subset of cells, e.g., after selecting the subset, using any method described herein.

The method may comprise selecting a subset of the plurality of cells that is enriched in G0 phase, G0/G1 phase, early G1 phase, G1 phase, late G1 phase, G1/S phase, S phase, G2/M phase, or M phase cells. In certain preferred embodiments, the method comprises selecting a subset of cells that is enriched in M phase cells. In certain embodiments, the methods may use differences between the plurality of cells, for example, the differences between the different phases of the cell cycle to separate or select the subset of the plurality of cells. In certain embodiments, the differences between the different phases of the cell cycle used to select the subset of may be physical (e.g., size of the cell, or amount of DNA in the cell). In certain embodiments, the differences between the different phases of the cell cycle used to separate or select the subset of the plurality of cells may be biochemical (e.g., cell surface markers expressed on the surface of the cells may be different at different cell cycles). In certain embodiments, the differences between the different phases of the cell cycle used to separate or select the subset of the plurality of cells may be physiological (e.g., cell adhesion to the surface).

The method may comprise contacting a population of cells with a dye, wherein selecting the subset of cells comprises selecting cells that comprise a similar amount of the dye. As used herein, the term “dye” refers to a molecule or particle that can absorb or emit light at an infrared, visible, or ultraviolet wavelength, such as a chromophore or fluorophore. For example, the dye may comprise fluorescein, Alexa Fluor® 488, phycoerythrin, R-phycoerythrin, Texas Red®, cyanine 5 (Cy5), bisbenzimidazole (Hoechst 33342), 4′,6-diamidino-2-phenylindole (DAPI), actinomycin, mithramycin, anthraquinone, TO-PRO-3, or propidium iodide. The dye may bind (e.g., specifically bind) to a cellular component, such as nucleic acids, double stranded nucleic acids, DNA, chromatin, or microtubules. The dye may bind to a molecule on the surface of a cell. The dye may be an intercalating agent, such as propidium iodide. In certain embodiments, cells at one stage of the cell cycle comprise more of a molecule that specifically binds to the dye (e.g., DNA or microtubules) than cells at a different stage of the cell cycle, so that the dye allows for the differentiation of cells at different stages. For example, dyes that bind to DNA allow for the differentiation of cells at different stages of the cell cycle because G2 and M phase cells contain twice as much DNA as G0 or G1 phase cells, and S phase cells contain an intermediate amount of DNA.

In some embodiments, the method comprise fluorescence activated cell sorting (FACS) (see, e.g., PCT Patent Application Publication Nos. WO 2014/109713 and WO 2010/118709, each of which is hereby incorporated by reference).

In some embodiments, the method does not comprise contacting the population of cells with a dye. For example, the forward scatter and side scatter channels of a FACS system may be used to differentiate cells at different stages of a cell cycle (e.g., “fluorescence activated cell sorting” may rely on forward scatter and side scatter rather than fluorescence). Similarly, methods such as centrifugation elutriation and mitotic shake-off do not rely on dyes.

Selecting a subset of the plurality of cells may comprise centrifugation elutriation (see, e.g., PCT Patent Application Publication Nos. WO 2013/067038 and WO 2003/093469; each of which is hereby incorporated by reference). A centrifugation elutriation system consists of a specialized centrifuge rotor in which the centrifugal force and opposing bulk medium flow create a gradient, with smaller cells at the top and larger cells at the bottom. The rotor speed or medium flow is manipulated such that the gradient of size-separated cells is pushed toward the top and the small cells at the top of the gradient are eventually pushed out of the elutriation chamber and into a collection vessel. With further manipulation of the rotor speed and medium flow, progressively larger cells are pushed out of the elutriation chamber. Since G1 cells are roughly half the size of mitotic or late G2 cells, centrifugal elutriation can be used to select cells according to their position in the cell cycle.

Selecting a subset of the plurality of cells may comprise mitotic shake-off (see, e.g., PCT Patent Application Publication No. WO 2010/118709; U.S. Pat. No. 5,710,022; and U.S. Patent Application Publication No. 2005/0273870; each of which is hereby incorporated by reference). Mitotic shake-off allows for the selection of spherical, mitotic (M) phase cells, which adhere less firmly to surfaces than G0 phase, G1 phase, S phase, and G2 phase cells. Thus, shaking cultures of adherent cells allows for the separation of M phase cells from cells at other phases.

### IV. Transforming a Cell

The method preferentially comprises transforming the cell or cells to generate the induced pluripotent stem cell(s). Transforming the cell or cells may comprise transforming the cell(s) with one or more proteins, one or more nucleic acids, one or more vectors, and/or one or more small molecules.

Generating an induced pluripotent stem cell may comprise transducing the cell with at least one gene selected from the group consisting of an Oct family gene, a Klf family gene, a Sox family gene, a Myc family gene, a Lin family gene, and a Nanog gene (see, e.g., U.S. Patent Application Publication No. 2009/0227032; hereby incorporated by reference). For example, generating an induced pluripotent stem cell may comprise transducing the cell with an Oct family gene, a Klf family gene, a Sox family gene, a Myc family gene, a Lin family gene, and a Nanog gene. The method may comprise transducing the cell with a gene for Kruppel-like factor 4 (Klf4), octamer-binding transcription factor 3/4 (Oct-3/4), octamer-binding transcription factor 4 (Oct-4), SRY (sex determining region Y)-box 2 (Sox2), L-Myc, N-Myc, and/or c-Myc. Generating a pluripotent stem cell may comprise transducing the cell with a gene for Oct3/4, Oct4, Klf4, Klf1, Klf2, Klf5, Sox2, Sox1, Sox3, Sox15, Sox17, Sox18, c-Myc, L-Myc, N-Myc, TERT, SV40 Large T antigen, HPV16 E6, HPV16 E7, Bmil, Lin28, Lin28b, Nanog, Glis1, Esrrb, and/or Esrrg. In certain preferred embodiments, generating a pluripotent stem cell comprises transducing the cell with a gene for Klf4, Oct-3/4, Oct-4, Sox2, L-Myc, N-Myc, and c-Myc. In certain embodiments, the method may comprise transducing the cell with at least a gene of the Sall1 and/or Sall4 gene family.

Transducing the cell may comprise transducing the cell with at least one vector, e.g., wherein the at least one vector comprises a gene for Klf4, Oct-3/4, Oct-4, Sox2, L-Myc, N-Myc, and/or c-Myc. The vector may comprise a plasmid, virus, transposable element, or nanoparticle. The vector may be, for example, a plasmid vector or a viral vector, such as a Sendai virus vector or an adenovirus vector. Transducing the cell may comprise transducing the cell with at least one Sendai virus vector, e.g., wherein the at least one Sendai virus vector comprises a gene for Klf4, Oct-3/4, Oct-4, Sox2, L-Myc, N-Myc, and/or c-Myc.

In certain embodiments, the method comprises transforming or contacting the cell with at least one gene product (e.g., a protein). For example, the at least one gene product may be a gene product of the Oct gene family, a gene product of the Klf gene family, a gene product of the Sox gene family, a gene product of the Myc gene family, a gene product of the Lin gene family, a gene product of the Sall1 gene family, a gene product of the Sall4 gene family, and a gene product of the Nanog gene. In certain embodiments, the method comprises transforming or contacting the cells with one or more gene products of each of the Oct gene family, the Klf gene family, the Sox gene family, the Myc gene family, the Lin gene family, the Sall1 gene family, the Sall4 gene family and the Nanog gene. In certain embodiments, the method comprises transforming or contacting the cells with one or more gene products of each of the Oct gene family, the Klf gene family, the Sox gene family, the Myc gene family, the Lin gene family, the Sall1 gene family, the Sall4 gene family and the Nanog gene, in combination with a cytokine. In certain embodiments, the cytokine is a fibroblast growth factor or a stem cell factor, for example.

Methods for generating pluripotent stem cells from a cell may include those described in U.S. Patent Application Publication Nos. 2011/0223669 and 2013/0065311; each of which is hereby incorporated by reference.

In some embodiments, the method comprises transforming the cell with at least one of a glycogen synthase kinase inhibitor, TGFP receptor inhibitor, cyclic AMP agonist, S-adenosyl homocysteine hydrolase inhibitor, and agent that promotes histone acetylation.

In some embodiments, the method comprises transforming the cell with a small molecule, such as valproic acid, BIX-01294, SB431412, or PD0325901. The method may comprise transforming the cell with valproic acid, BIX-01294, SB431412, and PD0325901. Methods for generating pluripotent stem cells from somatic cells using small molecules rather than nucleic acids include those described in PCT Patent Application Publication No. WO 2015/003643 (incorporated by reference) and Hou, P. et al, Science 341:651-54 (2013). One or more small molecules may be used instead of or in combination with one or more of the genes described herein, or the gene products thereof.

In certain embodiments, the method does not require gene integration. In some embodiments, the method comprises transforming the cell with one or more microRNAs (see, e.g., U.S. Pat. No. 8,852,941; hereby incorporated by reference). In certain embodiments, the one or more microRNAs facilitate the expression of at least one gene selected from the group comprising of Nanog, Sox2, Oct3/4, Klf4, Lin 28, L-Myc, N-Myc, and c-Myc. For example, the microRNAs may include microRNAs that suppress differentiation, microRNAs that promote dedifferentitation, microRNAs that suppress apoptosis, and microRNAs that control cell to cell adhesion.

In some embodiments, the method comprises differentiating the induced pluripotent stem cell(s). The method may comprise differentiating the iPSC(s) into fibroblast(s), B cell(s), T cell(s), hematopoietic cell(s), macrophage(s), monocyte(s), mononuclear cell(s), dendritic cell(s), myocyte(s), keratinocyte(s), melanocyte(s), adipocyte(s), epithelial cell(s), epidermal cell(s), chondrocyte(s), neural cell(s), glial cell(s), astrocyte(s), cardiac cell(s), cardiomyocyte(s), esophageal cell(s), gastric cell(s), pancreatic cell(s), hepatocyte(s), cumulus cell(s), or gametocyte(s). Methods for differentiating stem cells, such as iPSCs, may include those described in U.S. Patent Application Publication No. 2009/0227032, hereby incorporated by reference.

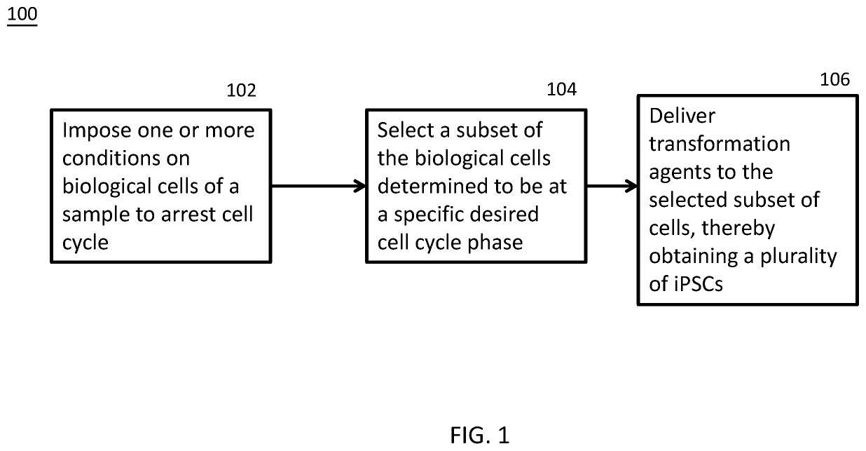

FIG. 1 is a block diagram showing a process (100) for generating a plurality of induced pluripotent stem cells (iPSCs) from a sample comprising a plurality of biological cells, according to an illustrative embodiment. In step 102, one or more conditions are imposed on the biological cells of the sample to arrest the cell cycle of the biological cells. Exemplary methods of arresting the cell cycle are described in section II of this Detailed Description, “Arresting the Cell Cycle”. In step 104, a subset of the biological cells that are determined to be at a specific desired cell cycle phase are selected. For example, cells in the G0 phase, G0/G1 phase, early G1 phase, G1 phase, late G1 phase, G1/S phase, S phase, G2/M phase, or M phase may be specifically selected. In certain embodiments, one or more methods described in section III of this Detailed Description, “Selecting Cells at a Phase of the Cell Cycle”, may be used to select the biological cells that are determined to be at a specific desired cell cycle phase. In step 106, transformation agents are delivered to the selected subset of cells to obtain the plurality of iPSCs. For example, exemplary methods of transforming a cell are described in section IV of this Detailed Description,

“Transforming a Cell”.

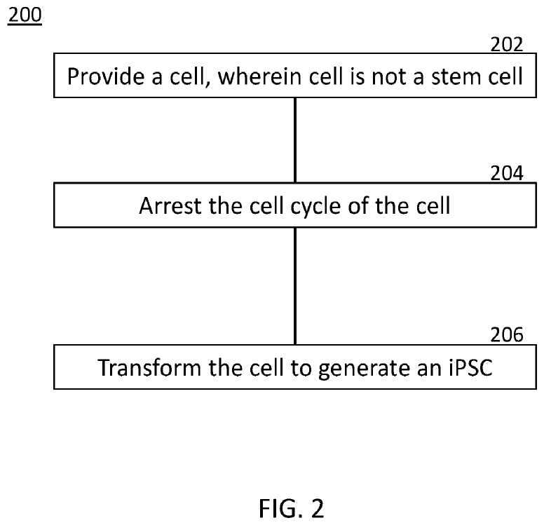

FIG. 2 is a block diagram showing a process (200) for generating an induced pluripotent stem cell (iPSC) from a cell. In step 202, a biological cell, which is not a stem cell is provided. For example, section I of this Detailed Description, “Cells”, describes different types of cell that may be used to generate an iPSC. In step 204, the cell cycle of the biological cell is arrested. Exemplary methods of arresting the cell cycle are described in section II of this Detailed Description, “Arresting the Cell Cycle”. For example, the cells may be arrested in the G0 phase, G0/G1 phase, early G1 phase, G1 phase, late G1 phase, G1/S phase, S phase, G2/M phase, or M phase. In step 206, the arrested cell is transformed to generate the iPSC. For example, exemplary methods of transforming a cell are described in section IV of this Detailed Description, “Transforming a Cell”.

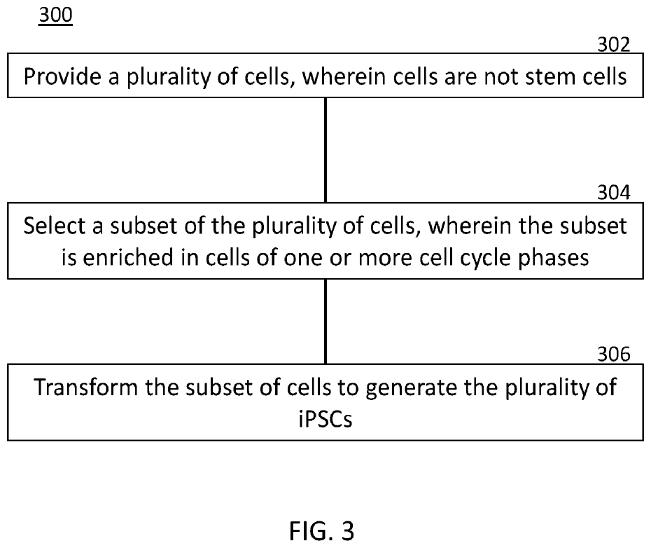

FIG. 3 is a block diagram showing a process (300) for generating a plurality of induced pluripotent stem cells (iPSCs) from a sample comprising a plurality of biological cells. In step 302, the plurality of biological cells, wherein the cells are not stem cells are provided. For example, section I of this Detailed Description, “Cells”, describes different types of cell that may be used to generate an iPSC. In step 304, a subset of the biological cells are selected, wherein the subset is enriched in cells of one or more cell cycles. Exemplary methods of arresting the cell cycle are described in section II of this Detailed Description, “Arresting the Cell Cycle”. In certain embodiments, one or more methods described in section III of this Detailed Description, “Selecting Cells at a Phase of the Cell Cycle”, may be used to select the biological cells that are determined to be at a specific desired cell cycle phase. For example, cells in the G0 phase, G0/G1 phase, early G1 phase, G1 phase, late G1 phase, G1/S phase, S phase, G2/M phase, or M phase may be specifically selected. In step 306, the selected subset of cells are transformed to generate the plurality of iPSCs. For example, exemplary methods of transforming cells are described in section IV of this Detailed Description, “Transforming a Cell”.

### EXEMPLIFICATION

### Example 1

### Harvesting Peripheral Blood Mononuclear Cells

A 12 mL LeucoSep™ tube is filled with 3 mL LeucoSep™ separation medium (Greiner Bio One). The tube is centrifuged for 30 seconds at 1000 rcf at room temperature to position the separation medium in the tube below the porous barrier.

4 mL of phosphate buffered saline (PBS; without calcium and magnesium) is added to a 15 mL conical tube. Human blood in a 4 mL vacutainer is inverted 10 times to mix the blood. The blood is then added to the conical tube containing the PBS, and the blood and PBS is mixed. The blood and PBS mixture is then poured into the LeucoSep™ tube.

The LeucoSep™ tube is centrifuged at room temperature for 30 minutes at 1250 rcf in a Labnet Centrifuge (or 2100 rpm in a Beckman swinging bucket centrifuge). The enriched cell fraction, containing lymphocytes and peripheral blood mononuclear cells, is collected by pouring off both the plasma supernatant and enriched cell fraction above the porous barrier into a new 15 mL centrifuge tube. The cells are pelleted at 500 rcf for 10 minutes in a Labnet centrifuge (or for 10 minutes at 1100 rpm in a Beckman centrifuge), and the supernatant is discarded.

The pellet is resuspended in 1 mL of freezing media (10% DMSO in heat-inactivated Fetal Bovine Serum). The 1 mL sample is divided into two 0.5 mL aliquots and frozen in a −80° C. freezer. Each 0.5 mL aliquot contains approximately 1,000,000 peripheral blood mononuclear cells.

### Example 2

### Transducing Peripheral Blood Mononuclear Cells

A 0.5 mL aliquot of peripheral blood mononuclear cells is washed with 0.5 mL of expansion media and placed in a 15 mL conical vial. The cells are pelleted at 250 rcf for 7 minutes, and the supernatant is decanted, leaving approximately 100 μL of media in the tube.

Transduction media is prepared, containing 0.4 mL StemPro-34 Lance Media; 5 μL hKOS; 5 μL hc-Myc; 3 μL h-Klf4; 2 μL Polybrene in water (1 mg/mL dilution); and Polybrene reagent (10 mg/mL).

Frozen CytoTune virus vials are placed in a 37° C. bath for 8 seconds, causing the reagent to melt, and then placed in a 4° C. cold block. The virus is mixed into the PBMC expansion media.

The transduction media is then placed in the 15 mL conical vial to resuspend the cell pellet. The transduction media and cells are placed in one well of a 24-well plate and incubated overnight at 37° C. in a humidified atmosphere of 5% CO₂.

### INCORPORATION BY REFERENCE

Each of the patents, published patent applications, and non-patent references cited herein are hereby incorporated by reference in their entirety.

### Equivalents

Those skilled in the art will recognize, or be able to ascertain using no more than routine experimentation, many equivalents to the specific embodiments of the invention described herein. Such equivalents are intended to be encompassed by the following claims.

1. A method of generating a plurality of induced pluripotent stem cells (iPSCs) from a sample comprising a plurality of biological cells, the method comprising:
(a) imposing one or more conditions on the biological cells of the sample for the purpose of arresting a cell cycle of the biological cells (e.g., a serum starvation condition; e.g., contact with a cell cycle inhibiting agent; e.g. incubation at a low temperature), wherein imposing the one or more conditions for arresting cell cycle comprises at least one of (i), (ii), and (iii):
(i) incubating the biological cells in a medium comprising at least one of (A), (B), and (C):
(A) a serum concentration that restricts cell growth [e.g., a serum concentration below a predetermined threshold concentration (e.g., less than 10%; e.g., less than about 5%; e.g., less than about 4%; e.g., less than about 3%; e.g., less than about 2%; e.g., less than about 1% serum concentration;); e.g., a serum concentration within a predetermined range (e.g., about 0% to about 10%; e.g., about 0.1% to about 5%; e.g., about 0.1% to about 2%; e.g., about 0.1% to about 1.0%); e.g., a serum free medium];
(B) a modified amino acid concentration that restricts cell growth [e.g., an amino acid concentration below a predetermined threshold concentration (e.g., less than 4 mM L-glutamine concentration); e.g., an amino acid concentration within a predetermined range]; and
(C) a concentration of one or more particular carbohydrates (e.g., glucose) that restricts cell growth [e.g., a concentration of one or more particular carbohydrates (e.g., glucose) below a predetermined threshold concentration (e.g., less than 5.5 mM glucose concentration); e.g., a concentration of one or more particular carbohydrates within a predetermined range] such that the cells are arrested in their cell cycle (e.g., by incubating the biological cells in the medium for a predetermined amount of time and/or until the cell cycles of the biological cells has been determined to have been arrested);
(ii) contacting the biological cells with a cell cycle inhibiting agent [e.g., a small molecule; e.g., a biomolecule; e.g., a reversible cell cycle inhibitor (e.g. the cell can restart its cell cycle after the agent is removed from media comprising the agent); e.g., a non-toxic agent (e.g., a concentration of the agent that is non-toxic to the cell is sufficient to arrest the cell cycle of the cell)] (e.g., by incubating the biological cells in a medium comprising the cell cycle inhibiting agent for a predetermined amount of time and/or until the cell cycles of the biological cells has been determined to have been arrested); and
(iii) incubating the biological cells at a low temperature [e.g., a temperature below a predefined threshold temperature (e.g., less than about 35° C., less than 34° C., less than 33° C., less than 32° C., less than 31° C., or less than 30° C.); e.g., a temperature within a given predefined temperature range; e.g., a temperature of about 27° C., about 28° C., about 29° C., about 30° C., about 31° C., about 32° C., or about 33° C.] for a predetermined amount of time and/or until the cell cycles of the biological cells has been determined to have been arrested;
(b) following step (a), selecting, from the biological cells, a subset of cells (e.g., separating the selected subset of cells from the remaining biological cells) determined [e.g., based on a property of the cells such as quantity of DNA within the cells, a size of the cells, a shape of the cells; e.g., via a cell sorting method (e.g., using a marker indicative of a specific cell cycle phase; e.g., using FACS, e.g., using centrifugal elutriation; e.g., using mitotic shake-off)] to be at a specific desired cell cycle phase, thereby obtaining a subset of cells that are enriched in cells at the same specific desired cell cycle phase (e.g., interphase; e.g., G0 phase; e.g., G0/G1 phase; e.g., early G1 phase; e.g., G1 phase; e.g., late G1 phase; e.g., G1/S phase; e.g., S phase; e.g., G2/M phase; e.g., M phase); and
(c) delivering to the selected subset of cells (e.g., having been separated from the remaining biological cells) one or more transformation agents (e.g., one or more transformation agents that, when delivered to a cell, transforms the cell into an iPSC), thereby obtaining a plurality of iPSCs.

2. The method of claim 1, comprising imposing the one or more conditions to arrest the cell cycle at a first cell cycle phase (e.g., G1/S phase, as in following contact with thymidine for 16-24 hours), and then removing the one or more conditions for a predefined amount of time (e.g., 12 hours) or until the specific desired cell cycle phase is determined to have been reached [e.g., (I) removing the one or more conditions on the cells (e.g., ceasing contact of the cells with thymidine) prior to selection of the subset of cells in step (b), or (II) continuing to impose the one or more conditions on the cells during the selection of the subset of cells in step (b) and removing the one or more conditions on the subset of cells following selection of the subset in step (b)], such that the selected subset of cells is in a second cell cycle phase (e.g., M phase; e.g., a second cell cycle phase different from the first cell cycle phase) when step (c) is performed.

3. The method of claim 2, wherein:
imposing the one or more conditions to arrest the cell cycle at the first cell cycle phase comprises incubating the biological cells in a first medium for a predetermined amount of time or until the first cell cycle phase has been determined to have been reached, and
removing the one or more conditions comprises at least one of (I) and (II):
(I) incubating the biological cells in a second medium for a predefined amount of time or until the second cell cycle phase is determined to have been reached, prior to selection of the subset of cells in step (b); and
(II) incubating the selected subset of cells in a second medium for a predefined amount of time or until the second cell cycle phase is determined to have been reached, following selection of the subset of cells in step (b).

4. The method of claim 3, wherein:
the first medium is a medium comprising at least one of (A), (B), and (C): (A) a serum concentration that restricts cell growth, (B) a modified amino acid concentration that restricts cell grown, and (C) a concentration of one or more particular carbohydrates (e.g., glucose) that restricts cell growth, and
the second medium is a medium comprising at least one of (A), (B), and (C): (A) a serum concentration that allows cell growth [e.g., a serum concentration that is higher than the serum concentration of the first medium; e.g., a serum concentration below a predetermined threshold concentration (e.g., greater than or equal to 10%); e.g., a serum concentration within a predetermined range (e.g., about 2% to about 35%; e.g., about 5% to about 30%; e.g., about 10% to about 25%), (B) an amino acid concentration that allows cell grown [e.g., an amino acid concentration that is higher than an amino acid concentration of the first medium; e.g., an amino acid concentration above a predetermined threshold concentration (e.g., greater than or equal to 4 mM L-glutamine concentration); e.g., an amino acid concentration within a predetermined range], and (C) a concentration of one or more particular carbohydrates (e.g., glucose) that allows cell growth [e.g., a glucose concentration that is higher than a glucose concentration of the first medium; e.g., a concentration of one or more particular carbohydrates (e.g., glucose) above a predetermined threshold concentration (e.g., greater than or equal to 5.5 mM glucose concentration); e.g., a concentration of one or more particular carbohydrates within a predetermined range].

5. The method of claim 3,
wherein the first medium comprises a cell cycle inhibiting agent at a concentration sufficient to arrest the cell cycle of the biological cells, and
wherein the second medium does not comprise the cell cycle inhibiting agent at a concentration sufficient to arrest the cell cycle of cells (e.g., the biological cells; e.g., the selected subset of cells).

6. The method of claim 1, wherein step (a) comprises contacting the biological cells with an agent for the purpose of modulating at least one pathway selected from the group consisting of a CEK interacting protein (cip) pathway, kinase inhibitory protein (kip) pathway, inhibitor of kinase 4 (INK4a) pathway, and an alternative reading frame (ARF) pathway.

7. The method of claim 6, wherein the agent modulates at least one protein selected from the group consisting of p14^{ARF}, p16^{INK4a}, p18, p19, p21, p27, p53, and p57.

8. The method of claim 6, wherein the agent comprises Transforming Growth Factor β (TGFβ).

9-15. (canceled)

16. The method of claim 1, wherein step (b) comprises:
contacting the plurality of biological cells with a labeled agent, the labeled agent comprising a detectable dye [e.g., a dye that absorbs light at one or more particular wavelengths (e.g., infrared wavelengths; e.g., visible wavelengths; e.g., ultraviolet wavelengths); e.g., a fluorescent dye that emits light at one or more particular wavelengths];
detecting a signal indicative of the presence and/or quantity of the labeled agent within and/or on the biological cells based on the detectable dye (e.g., detecting absorption of light by the biological cells at one or more particular wavelengths at which the dye absorbs light; e.g., detecting a fluorescent signal emitted from the biological cells); and
selecting, as the subset of cells, biological cells that are determined to comprise a substantially similar quantity of the labeled agent based on the detected signal (e.g., selecting cells for which a similar absorption signal is detected; e.g., selecting cells for which a similar fluorescent signal is detected).

17. The method of claim 16, wherein the labeled agent binds to nucleic acids (e.g., double stranded nucleic acids; e.g., DNA).

18. The method of claim 16, wherein the labeled agent binds to chromatin.

19. The method of claim 16, wherein the labeled agent binds to microtubules.

20. The method of claim 16, wherein the labeled agent binds to a cell surface marker (e.g., a particular protein on a surface of the cell).

21. The method of claim 16, wherein the labeled agent binds to or comprises a cellular marker for proliferation.

22. The method of claim 16, wherein the labeled agent binds to Ki-67 (e.g., wherein the labeled agent comprises an antibody that binds to Ki-67) [e.g., wherein Ki67 is preferentially expressed during the M phase, late G1 phase, S phase, and/or G2 phase, while cells in the G0 phase (non-cycling) have low or no Ki67 expression, wherein the labeled agent comprises an antibody that binds to (e.g., recognizes) Ki-67, thereby identifying cells that are in the M phase, late G1 phase, S phase, and/or G2 phase, as opposed to the G0 (non-cycling) phase].

23. The method of claim 16, wherein the labeled agent binds to histone H3 pSer28 (e.g., wherein the labeled agent comprises an antibody that binds to histone H3 pSer28).

24. The method of claim 16, wherein the labeled agent binds to Bromodeoxyuridine (BrdU) (e.g., wherein the labeled agent comprises an antibody that binds to BrdU; e.g., wherein BrdU is introduced into the biological cells such that replicating cells incorporate BrdU into newly synthesized DNA during replication, such that binding of the labeled agent to BrdU is indicative of cells that are replicating (e.g., cells that are in S phase)).

25. The method of claim 16, wherein the labeled agent comprises a dye that is initially nonfluorescent and becomes florescent following cleavage esterase within a cell, thereby allowing detection of cellular proliferation (e.g., wherein the labeled agent comprises Violet Proliferation Dye 450).

6. The method of claim 16, wherein the labeled agent comprises an antibody that binds to cyclin E thereby identifying cells that are in the S-phase.

27. The method of claim 16, wherein the labeled agent comprises an antibody that binds to cyclin B1 thereby identifying cells that are in (or moving towards) the G2/M-phase.

28-46. (canceled)
